(19) United States
(12) Patent Application Publication
(10) Pub. No.: US 2015/0118743 A1
(43) Pub. Date: Apr. 30, 2015

(54) NUCLEIC ACID EXTRACTION DEVICE

(71) Applicant: Seiko Epson Corporation, Tokyo (JP)

(72) Inventors: Masato HANAMURA, Shiojiri (JP); Kotaro IDEGAMI, Chino (JP)

(21) Appl. No.: 14/524,402

(22) Filed: Oct. 27, 2014

(30) Foreign Application Priority Data
Oct. 28, 2013 (JP) 2013-223011

(51) Int. Cl. C12N 15/10; B01L 3/00

(52) U.S. Cl. CPC C12N 15/1003; B01L 3/502784; B01L 2300/123; B01L 2300/0838; B01L 2300/087; B01L 2400/0677; B01L 2400/0481; B01L 2300/042; B01L 2200/0673; B01L 2200/0631
USPC 435/293.1; 422/502

(57) ABSTRACT

A nucleic acid extraction device has: a tube extending in a longitudinal direction in which a first plug formed of a wax or an oil, a second plug formed of a first washing liquid, a third plug formed of a wax, a fourth plug formed of an eluate, and a fifth plug formed of a wax or an oil are arranged in this order.

### BACKGROUND

1. Technical Field

The present invention relates to a nucleic acid extraction device.

2. Related Art

In recent years, medical care using genes, such as gene diagnosis and gene therapy, has attracted attention due to the development of techniques for using genes. In the agriculture and stockbreeding field, many methods using genes have also been developed for variety determination and breeding. A technique such as polymerase chain reaction (PCR) is widely used as a technique for using genes. Nowadays, PCR is an essential technique in analyzing the information of a biological substance. PCR is a method of applying a thermal cycle to a solution (reaction liquid) containing a nucleic acid (target nucleic acid) which is an amplification object and a reagent to amplify the target nucleic acid. As for the thermal cycle of the PCR, a method of applying a thermal cycle at two- or three-stage temperatures is generally used.

Currently, a simple examination kit such as an immunochromatography kit is mainly used for diagnosis of infections represented by influenza in the medical practice. However, in such a simple examination, the accuracy thereof may be insufficient, and PCR with which higher examination accuracy can be expected is desired to be applied to the diagnosis of infections. In general outpatient practices and the like in medical institutions, the time which can be spent for examination is limited to a short time since the time for diagnosis is limited. Therefore, for example, identification of influenza has been performed at the sacrifice of examination accuracy to reduce the time with simple examination such as immunochromatography.

Due to such a circumstance, it is necessary to reduce the time required for reaction in order to realize the examination by PCR with which higher accuracy can be expected. For example, JP-A-2009-136250 discloses, as an apparatus for causing a PCR in a short time, a biological sample reactor in which a chip for a biological sample reaction filled with a reaction liquid and a liquid which does not mix with the reaction liquid and has a lower specific gravity than the reaction liquid is rotated around a horizontal rotary shaft to move the reaction liquid, thereby applying a thermal cycle (JP-A-2009-136250). In addition, a method using magnetic beads (JP-A-2009-207459), as a droplet moving section to move droplets in a temperature changeable area on a substrate, thereby applying a thermal cycle of PCR (JP-A-2008-012490), and the like are disclosed as one method for PCR.

### SUMMARY

An advantage of some aspects of the invention is that it provides a nucleic acid extraction device which is capable of reducing a time required for isolation of a nucleic acid for nucleic acid amplification and has a good preserving property.

An aspect of the invention is directed to a nucleic acid extraction device including: a tube portion having a longitudinal direction in which a first plug formed of a wax or an oil, a second plug formed of a first washing liquid, a third plug formed of a wax, a fourth plug formed of an eluate, and a fifth plug formed of a wax or an oil are arranged in this order. A sixth plug formed of a second washing liquid and a seventh plug formed of a wax or an oil may be further provided from the side of the third plug between the third plug and the fourth plug. The wax may be a solid paraffin. The wax may have a melting point of 41° C. or higher. Here, the seventh plug is preferably a wax. In addition, the first plug and the fifth plug are preferably waxes. The tube portion may have an open end on the side of the fifth plug, and in that case, a detachable cock which seals the end on the side of the fifth plug of the tube portion may be further provided.

In one aspect of the invention, a detachable container may be further provided at an end on the side of the first plug of the tube portion, and the inside of the container and the inside of the tube portion may communicate with each other. The container may be flexible, and the inside of the tube portion may be pressurized by deforming the container in a state in which the container is connected to the tube portion.

In one aspect of the invention, a liquid reservoir may be provided at an end on the side of the first plug of the tube portion. A detachable cock which seals an end on the side of the liquid reservoir may be further provided. The liquid reservoir may be flexible, and the inside of the tube portion may be pressurized by deforming the liquid reservoir in a state in which the liquid reservoir is sealed with the cock.

In the nucleic acid extraction device according to any one of the aspects described above, the eluate may contain at least one of the group consisting of a reverse transcription enzyme, dNTP, and a primer for reverse transcription. The eluate preferably has a volume of 0.5 μL to 3.0 μL. The tube portion preferably has an internal diameter of 0.5 mm to 3.0 mm.

### BRIEF DESCRIPTION OF THE DRAWINGS

Embodiments of the invention will be described with reference to the accompanying drawings, wherein like numbers reference like elements.

FIG. 1 is a diagram schematically illustrating a main part of a nucleic acid extraction device according to an embodiment.

FIG. 2 is a diagram schematically illustrating a main part of a nucleic acid extraction device according to an embodiment.

FIG. 3 is a diagram schematically illustrating a main part of a nucleic acid extraction device according to an embodiment.

FIG. 4 is a diagram schematically illustrating a nucleic acid extraction device according to an embodiment.

FIG. 5 is a diagram schematically illustrating a nucleic acid extraction device according to an embodiment.

FIG. 6 is a diagram schematically illustrating a main part of a nucleic acid extraction device according to an embodiment.

FIG. 7 is a diagram schematically illustrating an example of a nucleic acid extraction kit according to an embodiment.

FIG. 8 is a diagram schematically illustrating an example of a nucleic acid extraction kit according to an embodiment.

FIG. 9 is a schematic diagram for illustrating a modification example of a nucleic acid extraction method according to an embodiment.

FIGS. 10A and 10B are diagrams schematically illustrating modification examples of a tube according to an embodiment.

FIG. 11 is a perspective view illustrating an example of a nucleic acid extraction apparatus according to an embodiment.

FIG. 12 is a perspective view illustrating an example of a nucleic acid extraction apparatus according to an embodiment.

FIG. 13 shows graphs illustrating results after preservation of a silicone oil and a solid paraffin for one week in Example 1.

FIG. 14 shows a graph illustrating results after preservation of a mineral oil for one week in Example 1.

FIG. 15 shows a graph illustrating results after preservation of the solid paraffin for two weeks in Example 1.

FIG. 16 shows a graph illustrating results after preservation of the solid paraffin for six weeks in Example 1.

FIG. 17A is a diagram schematically illustrating an example of a nucleic acid extraction device (A) used in Example 2.

FIG. 17B is a diagram schematically illustrating an example of a nucleic acid extraction device (B) used in Example 2.

FIG. 18 shows graphs illustrating results of RT-PCR using the nucleic acid extraction device according to the invention in Example 2.

### DESCRIPTION OF EXEMPLARY EMBODIMENTS

Hereinafter, several embodiments of the invention will be described. The embodiments to be described below are provided to describe examples of the invention. The invention is not limited to the following embodiments, and includes various modifications implemented without changing the gist of the invention. The elements of the entire configuration to be described below are not necessarily essential constituent elements of the invention.

### 1. Nucleic Acid Extraction Device

A nucleic acid extraction device 1000 of this embodiment has a tube portion 100, a first plug 10, a second plug 20, a third plug 30, a fourth plug 40, and a fifth plug 50.

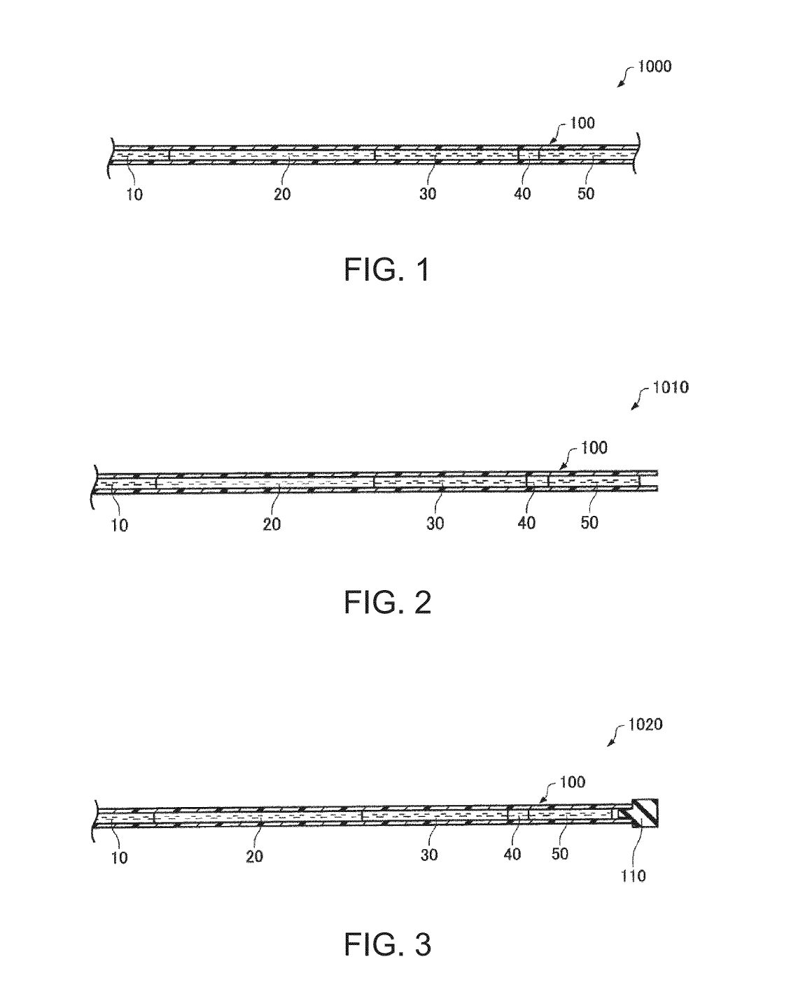

FIG. 1 is a diagram schematically illustrating a main part of the nucleic acid extraction device 1000 of this embodiment.

### 1.1. Tube Portion

The tube portion 100 constitutes a main part of the nucleic acid extraction device 1000. The nucleic acid extraction device 1000 may include various configurations other than the tube portion 100. Although not illustrated in FIG. 1, the nucleic acid extraction device 1000 may include a pipe, a container, a cock, a joint, a pump, a controller, and the like connected to the tube portion 100.

The tube portion 100 is a tubular part which has a cavity therein and allows a liquid to flow in the cavity in a longitudinal direction. The tube portion 100 extends in the longitudinal direction, but may be bent. The cavity in the tube portion 100 is not particularly limited in size and shape as long as the liquid can maintain a plug shape in the tube portion 100. The size of the internal cavity and the shape of a cross-section perpendicular to the longitudinal direction of the tube portion 100 may be changed in the longitudinal direction of the tube portion 100. Whether the liquid can maintain a plug shape in the tube portion 100 depends on conditions such as a material of the tube portion 100 and the kind of liquid, and thus the shape of the cross-section perpendicular to the longitudinal direction of the tube portion 100 is appropriately designed within a range in which the liquid can maintain a plug shape in the tube portion 100.

The shape of the cross-section perpendicular to the longitudinal direction of the external form of the tube portion 100 is also not limited. The thickness (a length from a side surface of the internal cavity to an external surface) of the tube portion 100 is also not particularly limited. When the cross-section perpendicular to the longitudinal direction of the internal cavity of the tube portion 100 has an annular shape, the internal diameter (a diameter of the circle of the cross-section perpendicular to the longitudinal direction of the internal cavity) of the tube portion 100 can be set to, for example, 0.5 mm to 3 mm. It is preferable that the internal diameter of the tube portion 100 be within this range since a liquid plug is easily formed with a wide range of material of the tube portion 100 and the kind of liquid.

The material of the tube portion 100 is not particularly limited, but for example, glass, a polymer, a metal or the like can be used. It is preferable that a material having transparency with respect to visible light, such as glass and a polymer, be selected as the material of the tube portion 100 since the inside (the inside of the cavity) can be observed from the outside of the tube portion 100. It is preferable that a substance transmitting a magnetic force or a non-magnetic body be selected as the material of the tube portion 100 since when magnetic particles are desired to pass through the tube portion 100, it is easily performed by giving a magnetic force from the outside of the tube portion 100.

In the tube portion 100, the first plug 10 formed of a wax or an oil, the second plug 20 formed of a first washing liquid, the third plug 30 formed of a wax, the fourth plug 40 formed of an eluate, and the fifth plug 50 formed of a wax or an oil are arranged in this order. Here, the wax is an organic substance which becomes a solid at room temperature, but becomes a liquid when being heated. The melting point of the wax which can be used is 31° C. or higher, preferably 36° C. or higher, more preferably 41° C. or higher, and even more preferably 46° C. or higher, and is 100° C. or lower, preferably 90° C. or lower, more preferably 80° C. or lower, and even more preferably 70° C. or lower. The wax is preferably formed of a neutral fat, a higher fatty acid, a hydrocarbon, and the like.

The wax is not particularly limited, and examples thereof include petroleum-derived waxes such as paraffin wax and microcrystalline wax, animal-derived waxes such as beeswax, wool wax, and spermaceti, vegetable-derived waxes such as carnauba wax, rosin wax, candelilla wax, and Japan wax, El Christa (registered trade name, Idemitsu Kosan Co., Ltd.), Nissan Electol (registered trade name, NOF CORPORATION), Poem (registered trade name, Riken Vitamin Co., Ltd.), Rikemal (registered trade name, Riken Vitamin Co., Ltd.), Neowax (registered trade name, Yasuhara Chemical Co., Ltd.), Hi-Wax (registered trade name, Mitsui Chemicals, Inc.), and Silicone Wax (registered trade name, Dow Corning Toray Co., Ltd.). As the oil, one selected from silicone oils such as a dimethylsilicone oil, paraffin oils, mineral oils, and mixtures thereof can be exemplified.

### 1.2. First Plug, Third Plug, and Fifth Plug

The first plug 10, the third plug 30, and the fifth plug 50 are each formed of a wax or an oil. The waxes of the first plug 10, the third plug 30, and the fifth plug 50 may be the same kind of wax or different kinds of waxes. When using an oil for the first plug 10 and the fifth plug 50, the same kind of oil or different kinds of oils may be used.

The second plug 20 is disposed between the first plug 10 and the third plug 30. Another liquid plug may be disposed in an area on the side of the first plug 10 opposite the second plug 20. It is preferable that bubbles and other liquids do not exist in the first plug 10. However, as long as particles adsorbing a nucleic acid can pass through the first plug 10, bubbles and other liquids may exist. In addition, it is preferable that bubbles and other liquids do not exist between the first plug 10 and the second plug 20. However, as long as particles adsorbing a nucleic acid can pass through from the first plug 10 to the second plug 20, bubbles and other liquids may exist. Similarly, it is preferable that bubbles and other liquids do not exist between the second plug 20 and the third plug 30. However, as long as particles adsorbing a nucleic acid can pass through from the second plug 20 to the third plug 30, bubbles and other liquids may exist.

The fourth plug 40 is disposed between the third plug 30 and the fifth plug 50. Another liquid plug may be disposed in an area on the side of the fifth plug 50 opposite the fourth plug 40. It is preferable that bubbles and other liquids do not exist in the third plug 30. However, as long as particles adsorbing a nucleic acid can pass through the third plug 30, bubbles and other liquids may exist. In addition, it is preferable that bubbles and other liquids do not exist between the third plug 30 and the fourth plug 40. However, as long as particles adsorbing a nucleic acid can pass through from the third plug 30 to the fourth plug 40, bubbles and other liquids may exist. Similarly, it is preferable that bubbles and other liquids do not exist between the fourth plug 40 and the fifth plug 50. However, as long as particles adsorbing a nucleic acid can pass through from the fourth plug 40 to the fifth plug 50, bubbles and other liquids may exist. Furthermore, it is preferable that bubbles and other liquids do not exist in the fifth plug 50.

The lengths of the first plug 10, the third plug 30, and the fifth plug 50 in the longitudinal direction of the tube portion 100 are not particularly limited within a range in which the plugs can be formed. The lengths of the first plug 10, the third plug 30, and the fifth plug 50 in the longitudinal direction of the tube portion 100 are specifically 1 mm to 50 mm. In order to keep a moving distance of particles from increasing excessively, the lengths are preferably 1 mm to 30 mm, and more preferably 5 mm to 20 mm. Among these, when the length of the third plug 30 in the longitudinal direction of the tube portion 100 is increased, it is possible to make it harder for the second plug 20 to be discharged when an aspect in which the fourth plug 40 is discharged from an end on the side of the fifth plug 50 (the downstream end) of the tube portion 100 is employed. In this case, a specific length of the third plug 30 can be set to 10 mm to 50 mm.

The first plug 10 and the fifth plug 50 function to prevent the first washing liquid (second plug 20) and the eluate (fourth plug 40) from being subjected to substance exchange with the outside air such as vaporization or from being contaminated from the outside even when at least one end of the tube portion 100 is open. Therefore, even when at least one end of the tube portion 100 is open to the outside air, the volumes of the first washing liquid and the eluate can be kept constant, and thus a fluctuation in the concentration of each liquid and contamination can be suppressed. Accordingly, it is possible to increase the accuracy of the concentrations of the nucleic acid and various agents in the nucleic acid extraction.

The third plug 30 functions to suppress the mixing of the first washing liquid (second plug 20) and the eluate (fourth plug 40). In addition, when the third plug 30 is formed of a wax having a higher viscosity, a “wiping effect” of the wax can be increased when particles are moved in an interface with the first washing liquid (second plug 20). Accordingly, when particles are moved from the first washing liquid plug which is the second plug 20 to the third wax plug 30, it is possible to make it harder for a water-soluble component adhering to the particles to be brought into the third plug 30 (wax).

### 1.3. Second Plug

The second plug 20 is disposed at a position between the first plug 10 and the third plug 30 in the tube portion 100. The second plug 20 is formed of a first washing liquid. The first washing liquid is a liquid which does not mix with either the wax or oil of the first plug 10 and the wax of the third plug 30. Examples of the first washing liquid include water and a buffer solution having a solute concentration of 10 mM or lower, preferably 7 mM or lower, and more preferably 5 mM or lower. The composition of the buffer solution is not particularly limited, but a tris-hydrochloric acid buffer solution and the like can be exemplified. An ethylenediaminetetraacetic acid (EDTA) or a surfactant may be contained. The first washing liquid preferably contains an alcohol such as ethanol in such an amount that adsorption of the nucleic acid to a carrier, a reverse transcription reaction, a PCR reaction, and the like are not inhibited. In this case, the concentration of the alcohol is not particularly limited. The alcohol concentration may be 70% or lower, 60% or lower, 50% or lower, 40% or lower, 30% or lower, 20% or lower, or 10% or lower, and is preferably 5% or lower or 2% or lower, more preferably 1% or lower or 0.5% or lower, and most preferably 0.2% or lower or 0.1% or lower. With such a first washing liquid, particles adsorbing a nucleic acid can be efficiently washed.

The volume of the second plug 20 is not particularly limited, and can be appropriately set with an amount of particles adsorbing a nucleic acid as an index. For example, when the volume of the particles is 0.5 μL, it is sufficient that the volume of the second plug 20 is 10 μL or greater, and it is preferably 20 μL to 50 μL, and more preferably 20 μL to 30 μL. When the volume of the second plug 20 is within this range, washing of the particles can be sufficiently performed when the volume of the particles is 0.5 μL. The greater the volume of the second plug 20, the more preferable to wash the particles. However, the volume of the second plug 20 can be appropriately set in consideration of the length and thickness of the tube portion 100, the length of the second plug 20 in the longitudinal direction of the tube portion 100 depending thereon, and the like.

The second plug 20 may be formed of a plurality of plugs partitioned by plugs formed of waxes or oils. When the second plug 20 is formed of a plurality of plugs partitioned by wax plugs or oil plugs, a plurality of first washing liquid plugs is formed. Accordingly, it is preferable that the second plug 20 be partitioned by wax plugs or oil plugs since when a washing target is a water-soluble substance, a concentration of the water-soluble substance reached by a partitioned first washing liquid is lower than a concentration of the water-soluble substance reached by an unpartitioned first washing liquid having the same volume. The number of plugs into which the second plug 20 is to be partitioned is arbitrarily set. When a washing target is a water-soluble substance, and for example, the second plug 20 is partitioned into two plugs having equal volumes, a calculated concentration of the water-soluble substance can be reduced up to ¼ of a concentration of a case in which the second plug 20 is not partitioned. The number of plugs into which the second plug is to be partitioned can be appropriately set in consideration of, for example, the length of the tube portion 100, the washing target, and the like.

### 1.4. Fourth Plug

The fourth plug 40 is disposed at a position between the third plug 30 and the fifth plug 50 in the tube portion 100. The fourth plug 40 is formed of an eluate.

The eluate refers to a liquid which eliminates a nucleic acid adsorbed to particles from the particles and elutes it in a liquid. Examples of the eluate include purified water such as sterilized water, distilled water, and ion-exchanged water and aqueous solutions in which at least one of an enzyme, dNTP, a probe, a primer, and a buffer is dissolved in such water. The eluate is a liquid which does not mix with either the wax of the third plug 30 and the wax or oil of the fifth plug 50.

When the eluate is water or an aqueous solution, the nucleic acid adsorbed to particles can be isolated (eluted) by immersing the particles adsorbing the nucleic acid in the eluate. When an aqueous solution in which at least one of an enzyme, dNTP, a probe, a primer, and a buffer is dissolved is selected as the eluate, the nucleic acid adsorbed to particles can be isolated (eluted), and some or all of components necessary for a reaction liquid of PCR can be contained in the eluate. Accordingly, the time and effort for a case in which the reaction liquid of PCR is prepared using the eluate can be further saved. The concentration when at least one of an enzyme, dNTP, a probe, a primer, and a buffer is dissolved in the eluate of the fourth plug 40 is not particularly limited and can be set according to the reaction liquid of PCR to be prepared.

Here, the dNTP represents four kinds of deoxyribonucleotide triphosphates (a mixture of deoxyadenosine triphosphate (dATP), deoxycytidine triphosphate (dCTP), deoxyguanosine triphosphate (dGTP), and thymidine triphosphate (dTTP)).

The volume of the fourth plug 40 is not particularly limited, and can be appropriately set with an amount of particles adsorbing a nucleic acid as an index. For example, when the volume of the particles is 0.5 μL, it is sufficient that the volume of the fourth plug 40 is 0.5 μL or greater, and it is preferably 0.8 μL to 5 μL, and more preferably 1 μL to 3 μL. When the volume of the fourth plug 40 is within this range, elution of the nucleic acid from the particles can be sufficiently performed when the volume of the particles is 0.5 μL. To elute the nucleic acid from the particles, the volume of the fourth plug 40 can be appropriately set in consideration of the length and thickness of the tube portion 100 and rapidity of a thermal cycle of PCR so that a thermal capacity of the reaction liquid is kept from increasing excessively.

### 1.5. Advantages

The nucleic acid extraction device 1000 of this embodiment has the tube portion 100 in which the waxes or oils, the first washing liquid, and the eluate are arranged in the form of plugs. Therefore, by introducing particles adsorbing a nucleic acid to the tube portion 100 from the side of the first plug 10 and by then moving the particles up to the fourth plug 40, the nucleic acid can be easily extracted in a very short time. Specifically, by: introducing the particles adsorbing the nucleic acid from the side of the first plug 10 of the tube portion 100; passing the particles through the wax or oil of the first plug 10; washing the particles with the first washing liquid of the second plug 20; and passing the particles through the wax of the third plug 30, the nucleic acid can be eliminated from the particles in the eluate of the fourth plug 40. That is, the nucleic acid extraction device 1000 of this embodiment can obtain an eluate containing the nucleic acid of high purity by moving the particles adsorbing the nucleic acid in the tube portion 100. Therefore, according to the nucleic acid extraction device 1000, the time and effort required for preprocessing for PCR can be significantly reduced.

In this device, waxes or oils are used as barrier layers, and thus even when one hollow tube is filled with preprocessing reagents, the reagents can be preserved while retaining desired functions. Particularly, when using waxes, the respective reagent components can maintain appropriate configurations and concentrations without being dispersed, and thus can be stably preserved for a long time. The effect of the wax is exhibited in all of the barrier layers, but is particularly strongly exhibited before the eluate (third plug) which is required to prevent ethanol from being brought thereinto.

### 1.6. Configuration of Nucleic Acid Extraction Device, Etc.

The nucleic acid extraction device of this embodiment has the tube portion 100, the first plug 10, the second plug 20, the third plug 30, the fourth plug 40, and the fifth plug 50. However, it may also include a configuration with other functions added thereto. The nucleic acid extraction device of this embodiment may include a combination of configurations to be described below, and modifications of the configurations.

### 1.6.1. End Portion of Tube Portion

FIG. 2 is a diagram schematically illustrating a nucleic acid extraction device 1010 which is a modification example of the nucleic acid extraction device. The nucleic acid extraction device of this embodiment may have, for example, an open end on the side of a fifth plug 50 of a tube portion 100. That is, as illustrated in FIG. 2, in the nucleic acid extraction device 1010, the end on the side of the fifth plug 50 of the tube portion 100 is open. According to the nucleic acid extraction device 1010, the fifth plug 50 and a fourth plug 40 can be discharged in order by applying a pressure to the inside of the tube portion 100 from a side of a first plug 10 of the tube portion 100. Accordingly, an eluate (fourth plug 40) containing a target nucleic acid can be easily dispensed to, for example, a reaction container for PCR using the nucleic acid extraction device 1010.

### 1.6.2. Cock

FIG. 3 is a diagram schematically illustrating a nucleic acid extraction device 1020 which is a modification example of the nucleic acid extraction device. The nucleic acid extraction device of this embodiment may further have, for example, a detachable cock 110 (stopper) to seal an end on the side of a fifth plug 50 of a tube portion 100 as illustrated in FIG. 3. The cock 110 can be made of, for example, a rubber, an elastomer, or a polymer. When the tube portion 100 is sealed by the cock 110, the cock 110 may be in contact with the fifth plug 50, or a gas such as air may be disposed between the fifth plug 50 and the cock 110. Although the cock 110 is detachable, the mechanism thereof is not particularly limited. The example of FIG. 3 illustrates an aspect in which a part of the cock 110 is inserted into the tube portion 100 and fixed, but the cock 110 may have a cap form.

When the cock 110 is removed in the nucleic acid extraction device 1020, the end on the side of the fifth plug 50 of the tube portion 100 is opened, and thus the aspect of the nucleic acid extraction device 1010 of FIG. 2 is provided, and an eluate (fourth plug 40) containing a target nucleic acid can be easily dispensed to, for example, a reaction container for PCR using the nucleic acid extraction device 1020. When the cock 110 seals the end on the side of the fifth plug 50 of the tube portion 100 (FIG. 3), an effect of suppressing the movement of each plug in the tube portion 100 is obtained. Thus, for example, when particles are moved in the tube portion 100, the movement of the plug with the movement of the particles can be suppressed.

### 1.6.3. Container

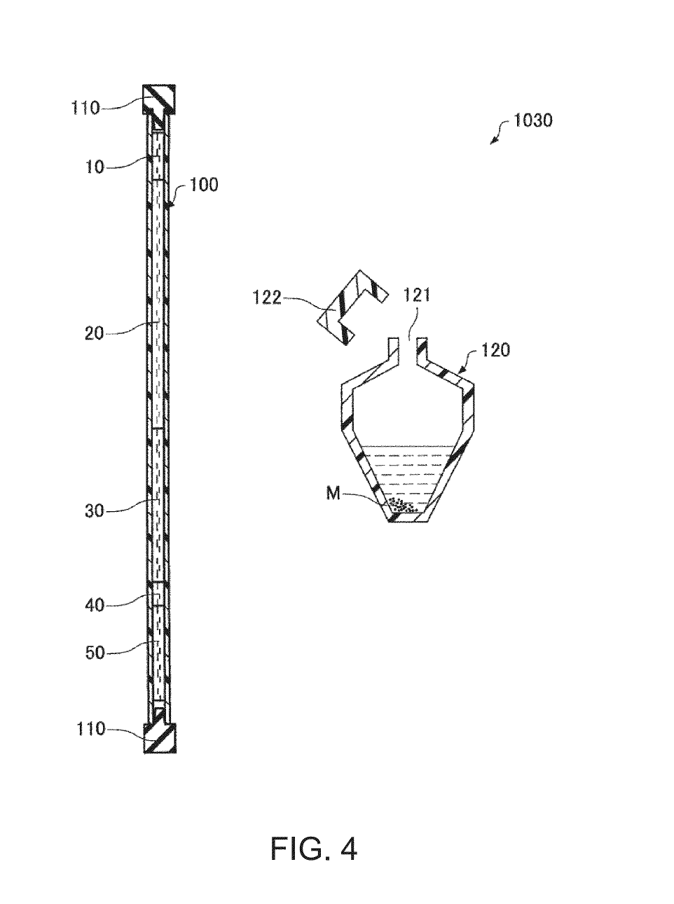

FIG. 4 is a diagram schematically illustrating a nucleic acid extraction device 1030 which is an example of the configuration of the nucleic acid extraction device. As illustrated in FIG. 4, the nucleic acid extraction device 1030 further has a detachable container 120 which can be connected to an end on the side of a first plug 10 (an upstream end) of a tube portion 100 by internal communication.

The container 120 can be formed as a separate member. The container 120 can accommodate a liquid therein. The container 120 has an opening 121 through which a liquid and a solid can be put in and out. The example of FIG. 4 illustrates an aspect in which the opening 121 of the container 120 is connected to the end on the side of the first plug 10 of the tube portion 100 by internal communication. The container 120 may have a plurality of openings 121. In this case, an aspect in which one opening 121 is connected to the end on the side of the first plug 10 of the tube portion 100 by internal communication may be employed.

The internal volume of the container 120 is not particularly limited. However, it can be set to 0.1 mL to 100 mL. If necessary, the opening 121 of the container 120 may have such a structure as to be sealed by a lid 122. The material of the container 120 is not particularly limited, and a polymer, a metal, and the like can be used.

The opening 121 of the container 120 can be connected to the end on the side of the first plug 10 of the tube portion 100. However, the connection between the container 120 and the tube portion 100 is not particularly limited as long as the contents do not leak therefrom. When the container 120 and the tube portion 100 are connected to each other, the inside of the container 120 and the inside of the tube portion 100 can be allowed to communicate with each other. If necessary, the container 120 can be detached from the tube portion 100.

As in the nucleic acid extraction device 1030, by providing the container 120, for example, particles, an adsorbent liquid, and a sample can be accommodated in the container 120 and a nucleic acid can be adsorbed to the particles. Thereafter, when the container 120 is connected to the end on the side of the first plug 10 of the tube portion 100, the particles can be easily introduced from the side of the first plug 10 of the tube portion 100 into the tube portion 100.

The adsorbent liquid refers to a liquid which becomes a site for adsorbing a nucleic acid to particles (magnetic particles M), and is, for example, an aqueous solution containing a chaotropic agent. The adsorbent liquid may contain a chelating agent, a surfactant, or the like, or may contain ethanol, methanol, acetonitrile, or the like. Specifically, in the adsorbent liquid, disodium dihydrogen ethylenediaminetetraacetate or its dihydrate may be dissolved, or polyoxyethylene sorbitan monolaurate or the like may be contained.

Here, the chaotropic agent refers to a substance which reduces an interaction between water molecules to make the structure of the water molecules unstable. Specific examples thereof include guanidinium ions, urea, and iodide ions. When the chaotropic agent exists in the water, the nucleic acid in the water has a thermodynamical advantage when existing by being adsorbed to a solid, rather than existing by being surrounded by water molecules, and thus the nucleic acid is adsorbed to surfaces of the particles. As a substance for generating the chaotropic agent in the water, guanidinium hydrochloride, sodium iodide, and the like are exemplified.

The container 120 can be shaken in a state of being disconnected from the tube portion 100 to sufficiently stir the liquid in the container 120. Accordingly, the nucleic acid can be rapidly adsorbed to the particles. The container 120 may have the lid 122 to seal the opening 121. Furthermore, by appropriately changing the amount of the sample to be introduced to the container 120 and the volume of the liquid (particularly, fourth plug 40) in the tube portion 100, the nucleic acid in the sample can be quantitatively concentrated in the eluate of the fourth plug 40.

When a flexible material such as a rubber, an elastomer, or a polymer is selected as the material of the container 120, the inside of the tube portion 100 can be pressurized by deforming the container 120 in a state in which the container 120 is connected to the tube portion 100. Thus, when the eluate of the fourth plug 40 is discharged from an end on the side of a fifth plug 50 of the tube portion 100, the pressure is easily applied from the side of the first plug 10 of the tube portion 100. Accordingly, the eluate can be dispensed to, for example, a reaction container for PCR.

### 1.6.4. Liquid Reservoir

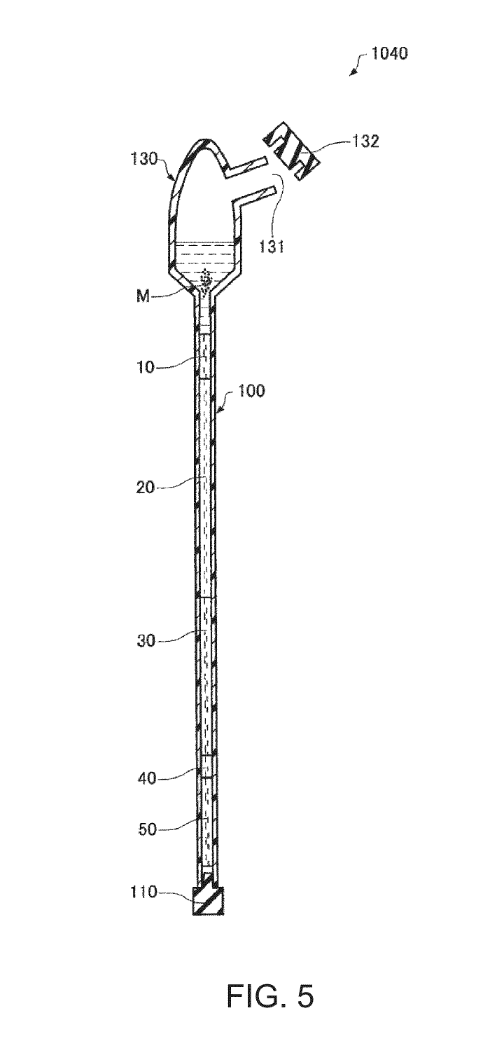

FIG. 5 is a diagram schematically illustrating a nucleic acid extraction device 1040 which is an example of the configuration of the nucleic acid extraction device. As illustrated in FIG. 5, the nucleic acid extraction device 1040 has a liquid reservoir 130 which is formed at an end on the side of a first plug 10 of a tube portion 100 to communicate with the tube portion 100. The inside of the liquid reservoir 130 and the inside of the tube portion 100 communicate with each other.

The liquid reservoir 130 can accommodate a liquid therein. The liquid reservoir 130 has an opening 131 through which a substance can be introduced from the outside to the inside of the liquid reservoir 130. The position at which the opening 131 is formed in the liquid reservoir 130 is not particularly limited. The liquid reservoir 130 may have a plurality of openings 131. The internal volume of the liquid reservoir 130 is not particularly limited. However, it can be set to 0.1 mL to 100 mL. The material of the liquid reservoir 130 is not particularly limited, and a polymer, a metal, or the like can be used. The material of the liquid reservoir 130 may be the same as that of the tube portion 100.

As in the nucleic acid extraction device 1040, by providing the liquid reservoir 130, for example, particles, an adsorbent liquid, and a sample can be accommodated in the liquid reservoir 130 and a nucleic acid can be adsorbed to the particles. The particles can be easily introduced into the tube portion 100 from the side of the first plug 10 of the tube portion 100.

In addition, the liquid reservoir 130 can be shaken together with the tube portion 100 to sufficiently stir the liquid in the liquid reservoir 130. Accordingly, the nucleic acid can be rapidly adsorbed to the particles. Furthermore, by appropriately changing the amount of the sample to be introduced to the liquid reservoir 130 and the volume of the liquid in the tube portion 100, the nucleic acid in the sample can be quantitatively concentrated in an eluate.

When the liquid reservoir 130 is provided as in the nucleic acid extraction device 1040, a detachable lid 132 may be further provided to seal the opening 131 of the liquid reservoir 130. When a flexible material such as a rubber, an elastomer, or a polymer is selected as the material of the liquid reservoir 130, the inside of the tube portion 100 can be pressurized by deforming the liquid reservoir 130 in a state in which the lid 132 is mounted on the liquid reservoir 130.

Thus, when the eluate of a fourth plug 40 in which the nucleic acid is eluted is discharged from an end on the side of a fifth plug 50 of the tube portion 100, the pressure can be easily applied from the side of the first plug 10 of the tube portion 100. Accordingly, it is possible to perform the processing ranging from a process of introducing a sample to the container 120 to a process of easily dispensing an eluate to, for example, a reaction container for PCR. In addition, when mounting the lid 132, it is possible to suppress liquid leakage when the liquid reservoir 130 is shaken together with the tube portion 100. Thus, the efficiency of adsorbing the nucleic acid to particles can be improved.

### 1.6.5. Sixth Plug and Seventh Plug

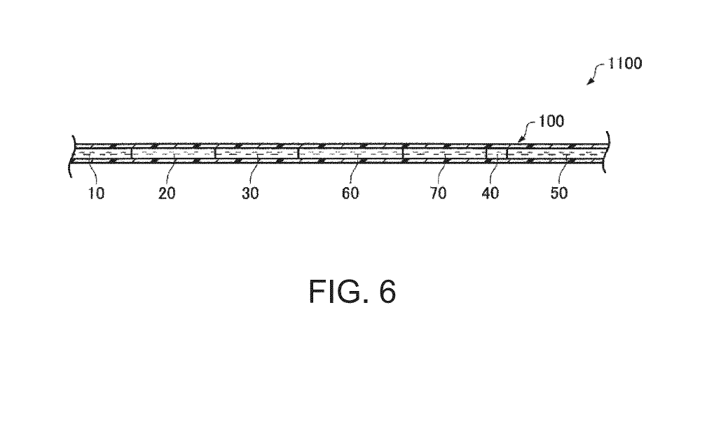

The nucleic acid extraction device of this embodiment may have a sixth plug and a seventh plug in the tube portion. FIG. 6 is a diagram schematically illustrating a nucleic acid extraction device 1100 having a sixth plug 60 and a seventh plug 70 in a tube portion 100.

The nucleic acid extraction device 1100 has a configuration in which between a third plug 30 and a fourth plug 40 in the tube portion 100 of the above-described nucleic acid extraction device, the sixth plug 60 formed of a second washing liquid which does not mix with a wax or an oil and the seventh plug 70 formed of a wax or an oil are added in order from the side of the third plug 30.

The sixth plug 60 is disposed at a position on the side of the third plug 30 in the tube portion 100 opposite a second plug 20. The sixth plug 60 is formed of a second washing liquid. The second washing liquid is a liquid which does not mix with both the wax of the third plug 30 and the wax or oil of the seventh plug 70. Examples of the second washing liquid include water and a buffer solution having a solute concentration of 10 mM or lower, preferably 7 mM or lower, and more preferably 5 mM or lower. The composition of the buffer solution is not particularly limited, but a tris-hydrochloric acid buffer solution and the like can be exemplified. An ethylenediaminetetraacetic acid (EDTA) and the like may be contained. The second washing liquid may have a composition which is the same as or different from that of the first washing liquid. It is preferable that the second washing liquid contain a citric acid and be acidic so that a pH thereof is approximately 4.0.

The volume of the sixth plug 60 is not particularly limited, and can be appropriately set with an amount of particles adsorbing a nucleic acid as an index. For example, when the volume of the particles is 0.5 μL, it is sufficient that the volume of the sixth plug 60 is 10 μL or greater, and it is preferably 20 μL to 50 μL, and more preferably 20 μL to 30 μL. When the volume of the sixth plug 60 is within this range, washing of the particles can be sufficiently performed when the volume of the particles is 0.5 μL. The greater the volume of the sixth plug 60, the more preferable to wash the particles. However, the volume of the sixth plug 60 can be appropriately set in consideration of the length and thickness of the tube portion 100, the length of the sixth plug 60 in the longitudinal direction of the tube portion 100 depending thereon, and the like.

The sixth plug 60 may be partitioned by wax or oil plugs so as to be formed of a plurality of plugs. When the sixth plug 60 is formed of a plurality of plugs partitioned by wax plugs or oil plugs, a plurality of second washing liquid plugs is formed. Accordingly, it is preferable that the sixth plug 60 be partitioned by wax plugs or oil plugs since when a washing target is a water-soluble substance, a concentration of the water-soluble substance reached by a partitioned second washing liquid is lower than a concentration of the water-soluble substance reached by an unpartitioned second washing liquid having the same volume. The number of plugs into which the sixth plug 60 is to be partitioned is arbitrarily set. When a washing target is a water-soluble substance, and for example, the sixth plug 60 is partitioned into two plugs having equal volumes, a calculated concentration of the water-soluble substance can be reduced up to ¼ of a concentration of a case in which the sixth plug 60 is not partitioned. The number of plugs into which the sixth plug is to be partitioned can be appropriately set in consideration of, for example, the length of the tube portion 100, the washing target, and the like. When the first washing liquid of the second plug 20 is the same as the second washing liquid of the sixth plug 60, similar effects to those of a case in which the second plug 20 is partitioned in a nucleic acid extraction device which does not have the above-described sixth plug 60 and seventh plug 70 are obtained.

The seventh plug 70 is formed of a wax or an oil which does not mix with the liquids of the sixth plug 60 and the fourth plug 40 next thereto. When the seventh plug 70 is a wax, it may be a different kind of wax from the waxes of the first plug 10, the third plug 30, and the fifth plug 50. Examples of the wax include those exemplified in the case of the first plug 10 and the like. When the seventh plug 70 is an oil, it may be a different kind of oil from the oils of the first plug 10 and the fifth plug 50. Examples of the oil include those exemplified in the case of the first plug 10 and the like. When a wax is used as the seventh plug 70, the reagents contained in the liquids of the fourth plug 40 and the sixth plug 60 can be further effectively prevented from being diffused through the seventh plug 70, and the respective reagent components can maintain appropriate configurations and concentrations.

It is preferable that bubbles and other liquids do not exist in the seventh plug 70. However, as long as particles adsorbing a nucleic acid can pass through the seventh plug 70, bubbles and other liquids may exist. In addition, it is preferable that bubbles and other liquids do not exist between the fourth plug 40 and the sixth plug 60 next to the seventh plug 70. However, as long as particles adsorbing a nucleic acid can be moved in the tube portion 100, bubbles and other liquids may exist. It is preferable that bubbles and other liquids do not exist in the seventh plug 70.

The length of the seventh plug 70 in the longitudinal direction of the tube portion 100 is not particularly limited within a range in which the plug can be formed. The length of the seventh plug 70 in the longitudinal direction of the tube portion 100 is specifically 1 mm to 50 mm. In order to keep a moving distance of particles from increasing excessively, the length is preferably 1 mm to 30 mm, and more preferably 5 mm to 20 mm. In the nucleic acid extraction device 1100, when the length of the seventh plug 70 in the longitudinal direction of the tube portion 100 is increased, it is possible to make it harder for the sixth plug 60 to be discharged when an aspect in which the fourth plug 40 is discharged from an end on the side of the fifth plug 50 of the tube portion 100 is employed. In this case, a specific length of the seventh plug 70 can be set to 10 mm to 50 mm.

In addition, the seventh plug 70 functions to suppress the mixing of the second washing liquid (sixth plug 60) and the eluate (fourth plug 40). In addition, when the seventh plug 70 is formed of a wax or an oil having a higher viscosity, a “wiping effect” of the wax or the oil can be increased when particles are moved in an interface with the second washing liquid (sixth plug 60). Accordingly, when particles are moved from the second washing liquid plug which is the sixth plug 60 to the seventh wax or oil plug 70, it is possible to make it harder for a water-soluble component adhering to the particles to be brought into the seventh plug 70 (wax or oil).

According to the nucleic acid extraction device 1100, particles adsorbing a nucleic acid can be washed in the second plug 20 and the sixth plug 60. Therefore, efficiency of washing the particles can be further increased.

In the nucleic acid extraction device 1100, the first washing liquid of the second plug 20 may contain a chaotropic agent. For example, when the first washing liquid of the second plug 20 contains guanidinium hydrochloride, it is possible to wash the particles while maintaining or strengthening the adsorption of the nucleic acid adsorbed to the particles in the second plug 20. The concentration when the second plug 20 contains guanidinium hydrochloride can be set to, for example, 3 mol/L to 10 mol/L, and preferably 5 mol/L to 8 mol/L. When the concentration of the guanidinium hydrochloride is within this range, it is possible to wash other foreign substances while more stably adsorbing the nucleic acid adsorbed to the particles.

When the second washing liquid of the sixth plug 60 is water or a buffer solution, it is possible to more stably adsorb the nucleic acid adsorbed to the particles and also possible to perform washing in the second plug 20 (first washing liquid), and it is possible to further wash the particles while diluting the chaotropic agent in the sixth plug 60 (second washing liquid).

It should be easily understood that the nucleic acid extraction device 1100 having the sixth plug 60 and the seventh plug 70 in the tube portion 100 may also have a configuration with the above-described cock, container, liquid reservoir, and the like added thereto, and thus similar effects to those described above are obtained.

### 2. Nucleic Acid Extraction Kit

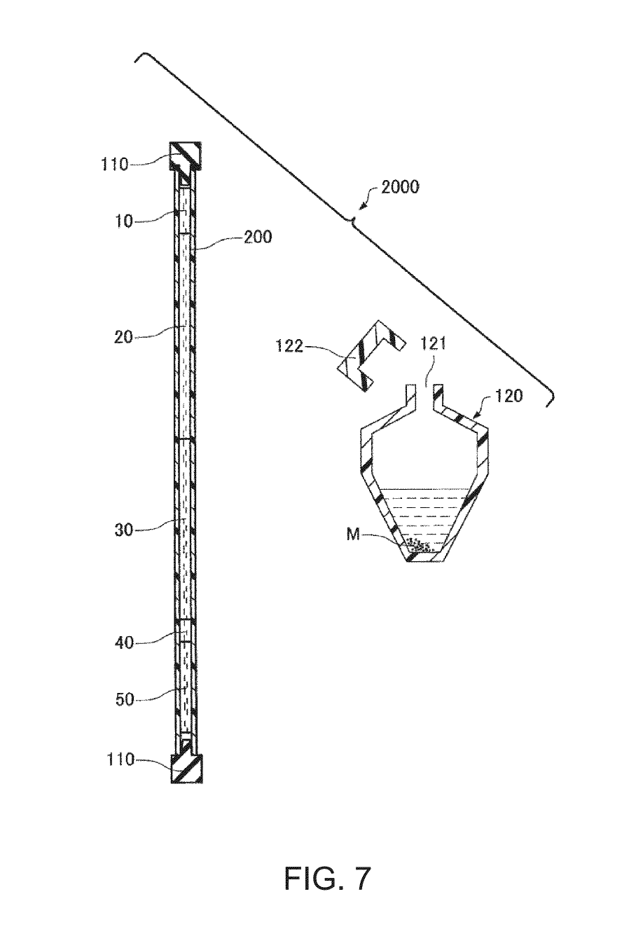

FIG. 7 is a schematic diagram illustrating an example of a nucleic acid extraction kit of this embodiment. A nucleic acid extraction kit 2000 illustrated in FIG. 7 includes components constituting the main part of the above-described nucleic acid extraction device. Similar configurations to those described in the clause “1. Nucleic Acid Extraction Device” will be denoted by the same reference numerals, and detailed description thereof will be omitted.

The nucleic acid extraction kit 2000 of this embodiment includes a tube 200 in which a first plug 10 formed of a wax or an oil, a second plug 20 formed of a first washing liquid which does not mix with a wax or an oil, a third plug 30 formed of a wax, a fourth plug 40 formed of an eluate which does not mix with a wax or an oil, and a fifth plug 50 formed of a wax or an oil are arranged in this order, and a container 120 which can be connected to an end on the side of the first plug 10 of the tube 200 by internal communication.

The tube 200 has an aspect in which both ends of the tube portion 100 of the nucleic acid extraction device 1000 are open. The tube 200 has a cavity therein and has a tubular shape in which a liquid can be allowed to flow in a longitudinal direction in the cavity. The internal shape, external shape, size, properties, material, and the like of the tube 200 are similar to those of the tube portion 100 of the nucleic acid extraction device 1000. The plugs arranged in the tube 200 are similar to the plugs arranged in the tube portion 100 of the nucleic acid extraction device 1000. Both ends of the tube 200 may be sealed by detachable cocks 110. When both the ends of the tube 200 are sealed by the cocks 110, for example, storage and transfer of the nucleic acid extraction kit 2000 are facilitated. Furthermore, when the cock 110 seals an end on the side of the fifth plug 50 of the tube 200 during the use of the tube 200, the movement of each plug in the tube 200 can be suppressed when particles are moved in the tube 200, and thus washing and extraction can be further facilitated. Since the cock 110 is detachable, the end on the side of the fifth plug 50 of the tube 200 can be opened, and thus the eluate of the fourth plug 40 in which a nucleic acid is eluted is easily discharged from the end on the side of the fifth plug 50 of the tube 200.

The container 120 is similar to the container 120 described in the clause of the nucleic acid extraction device 1000.

In the example of FIG. 7, both the ends of the tube 200 are sealed by the detachable cocks 110. The nucleic acid extraction kit 2000 may include a lid 122 which detachably seals an opening 121 of the container 120, or the opening 121 of the container 120 may be sealed by the detachable lid 122. The nucleic acid extraction kit 2000 may accommodate some or all of components of an adsorbent liquid in the container 120.

In addition, in the nucleic acid extraction kit 2000, the container 120 may accommodate an adsorbent liquid and magnetic particles. Thus, a process of adsorbing, when a sample is introduced into the container 120, a nucleic acid contained in the sample to the magnetic particles can be performed in the container 120. Accordingly, it is possible to more rapidly perform preprocessing of PCR without the need to provide another container. In addition, in this case, the opening 121 of the container 120 may be sealed by the detachable lid 122 if necessary. The magnetic particles will be described later in detail.

When the container 120 is made of a flexible material as described above, the inside of the tube 200 can be pressurized by deforming the container 120 in a state in which the container 120 is connected to the tube 200. Thus, when the eluate of the fourth plug 40 in which the nucleic acid is eluted is discharged from the end on the side of the fifth plug 50 of the tube 200, the pressure can be easily applied from the side of the first plug 10 of the tube 200. Accordingly, it is possible to easily dispense the eluate to, for example, a reaction container for PCR.

The nucleic acid extraction kit 2000 may include other configurations such as a cock, a lid, an instruction manual, a reagent, and a case, other than the tube 200 and the container 120. Here, an example has been shown in which five plugs are arranged in the tube 200. However, as described in the clause “1.6. Nucleic Acid Extraction Device”, it should be easily understood that if necessary, other plugs such as the sixth plug 60 and the seventh plug 70 may be arranged in the tube 200 (tube portion 100).

The nucleic acid extraction kit 2000 of this embodiment has the container 120 which can be connected to the end on the side of the first plug 10 of the tube 200 by internal communication. Accordingly, when particles and a sample are accommodated in the container 120, the nucleic acid can be adsorbed to the particles, and thus the particles can be easily introduced into the tube 200 from the side of the first plug of the tube 200 when the container 120 is connected to the end on the side of the first plug 10 of the tube 200. In addition, since the nucleic acid extraction kit 2000 of this embodiment has the container 120, the container 120 can be shaken, and thus the liquid in the container 120 can be sufficiently stirred. Accordingly, the nucleic acid can be rapidly adsorbed to the particles.

When connecting the container 120 to the tube 200, the particles adsorbing the nucleic acid are easily moved up to the fourth plug 40 by introducing the particles from the end on the side of the first plug 10 of the tube 200. Accordingly, the nucleic acid can be easily extracted in a very short time. The nucleic acid extraction kit 2000 can obtain an eluate containing the nucleic acid of high purity by moving the particles adsorbing the nucleic acid in the tube 200. Therefore, according to the nucleic acid extraction kit 2000, the time and effort required for preprocessing for PCR can be significantly reduced.

### 3. Nucleic Acid Extraction Method

All of the nucleic acid extraction device, the nucleic acid extraction kit, and the modifications thereof, which have been described above, and a nucleic acid extraction apparatus to be described later can be appropriately used in a nucleic acid extraction method of this embodiment. Hereinafter, a method using the above-described nucleic acid extraction kit 2000 will be described as an example of the nucleic acid extraction method of this embodiment.

The nucleic acid extraction method of this embodiment includes: a process of introducing a sample containing a nucleic acid to the flexible container 120 accommodating magnetic particles M and an adsorbent liquid; a process of adsorbing the nucleic acid to the magnetic particles M by vibrating the container 120; a process of connecting the container 120 to the end on the side of the first plug 10 of the tube 200, in which the first plug 10 formed of a wax or an oil, the second plug 20 formed of a first washing liquid which does not mix with a wax or an oil, the third plug 30 formed of a wax, the fourth plug 40 formed of an eluate which does not mix with a wax or an oil, and the fifth plug 50 formed of a wax or an oil are arranged in this order, by allowing the inside of the container 120 and the inside of the tube 200 to communicate with each other; a process of passing the magnetic particles M through the tube 200 from the container 120 by applying a magnetic force to move the magnetic particles M up to the position of the fifth plug 50; and a process of eluting the nucleic acid from the magnetic particles M in the eluate of the fourth plug 40.

In the nucleic acid extraction method of this embodiment, various particles (for example, silica particles, polymer particles, magnetic particles, or the like) can be used as long as the particles can adsorb the nucleic acid using an adsorbent liquid and can be moved in the tube 200. However, in an embodiment of the nucleic acid extraction method to be described below, the magnetic particles M which are particles containing a magnetic body and can adsorb the nucleic acid to surfaces of the particles are used. When particles other than the magnetic particles M are moved in the tube, this movement can be performed using, for example, gravity or a potential difference.

In the nucleic acid extraction method of this embodiment, a material transmitting a magnetic force is selected for the container 120 and the tube 200, and a magnetic force is applied from the outside of the container 120 and the tube 200 to move the magnetic particles M in the container 120 and the tube 200.

The sample contains a nucleic acid which becomes a target. Hereinafter, it may be simply referred to as a target nucleic acid. The target nucleic acid is, for example, DNA and/or RNA (DNA: Deoxyribonucleic Acid, and/or RNA: Ribonucleic Acid). The target nucleic acid is extracted from the sample through the nucleic acid extraction method of this embodiment, is eluted in the eluate, and is then used as, for example, a template of PCR. Examples of the sample include blood, nasal cavity mucous, oral mucous membrane, and other various biological samples.

### 3.1. Process of Introducing Sample to Container

The process of introducing the sample to the container 120 can be performed in such a manner that for example, the sample is attached to a swab and the swab is put from the opening 121 of the container 120 to immerse the swab in the adsorbent liquid. The sample may be introduced from the opening 121 of the container 120 using a pipette or the like. When the sample has a paste form or a solid form, the sample may be attached or fed to an internal wall of the container 120 using a spoon, tweezers, or the like from the opening 121 of the container 120.

### 3.2. Process of Adsorbing Nucleic Acid to Magnetic Particles

The process of adsorbing the nucleic acid is performed by vibrating the container 120. When there is the lid 122 which seals the opening 121 of the container 120, this process can be more efficiently performed by sealing the container 120 using the lid 122. Through this process, the target nucleic acid is adsorbed to surfaces of the magnetic particles M by an action of a chaotropic agent. In this process, a nucleic acid other than the target nucleic acid or protein may be adsorbed to the surfaces of the magnetic particles M.

As the method of vibrating the container 120, an apparatus such as a vortex shaker may be used, or the container 120 may be shaken by an operator's hand. A magnetic property of the magnetic particles M may be used to vibrate the container 120 while giving a magnetic field from the outside. The time for vibration of the container 120 can be appropriately set. For example, when the shape of the container 120 is roughly a cylindrical shape having a diameter of approximately 20 mm and a height of approximately 30 mm, stirring is sufficiently performed by shaking and vibrating the container 120 for 10 seconds by hand to adsorb the nucleic acid to the surfaces of the magnetic particles M.

### 3.3. Process of Connecting Container to Tube

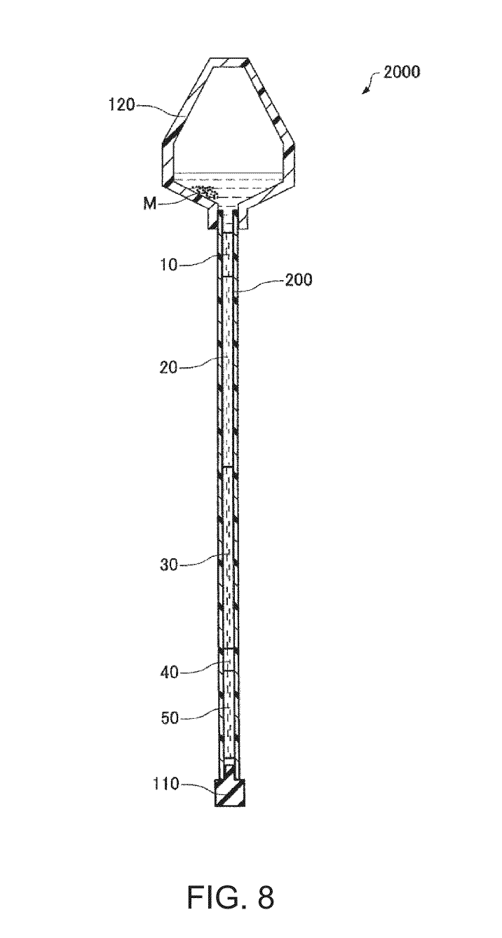

Next, the container 120 is connected to the end on the side of the first plug 10 of the tube 200 as illustrated in FIG. 8. Each plug in the tube 200 is difficult to move in the tube 200 since the cock 110 on the side of the seventh plug 70 is not removed even when the cock 110 on the side of the first plug 10 is removed. When the cock 110 is attached to the end on the side of the first plug 10 of the tube 200, this process is performed after removing the cock 110. The container 120 and the tube 200 are connected to each other so that the contents do not leak therefrom, and are thus allowed to communicate with each other so that the contents can be allowed to flow between the inside of the container 120 and the inside of the tube 200.

### 3.4. Process of Moving Magnetic Particles

Through the above-described processes, the magnetic particles M adsorbing the nucleic acid in the container 120 enter into a state in which these can be allowed to flow in the tube 200. As the method of introducing the magnetic particles M adsorbing the nucleic acid to the tube 200, a method using gravity or a centrifugal force may be used. The introduction method is not particularly limited, but in this embodiment, the introduction is performed by applying a magnetic force from the outside of the container 120 and the tube 200. The magnetic force can be applied using, for example, a permanent magnet, an electromagnet, or the like. However, it is preferable that the magnetic force be applied using a permanent magnet in view of no generation of heat and the like. When a permanent magnet is used, the introduction may be performed by moving the magnet by an operator's hand, or a mechanical device or the like may be used. The magnetic particles M have such a property as to be pulled by a magnetic force. Accordingly, using this property, the relative arrangement between: the container 120 and the tube 200; and the position of the permanent magnet is changed to move the magnetic particles M from the inside of the container 120 to the tube 200. Thus, the magnetic particles M are moved from the first plug 10 to the fourth plug 40 through the plugs in order. A staying time in each plug when the magnetic particles M pass through each plug is not particularly limited, and the magnetic particles M may be moved to reciprocate in the longitudinal direction of the tube 200 in the same plug.

### 3.5. Process of Eluting Nucleic Acid

When the magnetic particles M reach the fourth plug 40, the nucleic acid adsorbed to the magnetic particles M is eluted to the eluate of the fourth plug 40 by the action of the eluate. Through this process, the nucleic acid is eluted from the sample to the eluate, and a state in which the nucleic acid is extracted from the sample is made.

### 3.6. Advantages

According to the nucleic acid extraction method of this embodiment, it is possible to easily extract a nucleic acid in a very short time. In the nucleic acid extraction method of this embodiment, magnetic particles M adsorbing a nucleic acid are moved in the tube 200, and thus it is possible to obtain an eluate containing the nucleic acid of high purity. According to the nucleic acid extraction method of this embodiment, the time and effort required for preprocessing for PCR can be significantly reduced.

3.7. Process of Discharging Fourth Plug from Tube

The nucleic acid extraction method of this embodiment may include a process of discharging the fifth plug 50 and the fourth plug 40 from an end of the tube 200 on the side opposite to the end connected to the container 120 by deforming the container 120.

This process can be performed by deforming the container 120 after the process described in the clause “3.5. Process of Eluting Nucleic Acid”. When the fourth plug 40 is discharged, the fifth plug 50 is discharged first. The cock 110 sealing the side of the fifth plug 50 of the tube 200 is removed prior to this process to open the end on the side of the fifth plug 50 of the tube 200.

When an external force is applied to the container 120 to increase the internal pressure and the container 120 is thus deformed, each plug is moved from the side of the first plug 10 to the side of the fifth plug 50 of the tube 200 due to the pressure. Accordingly, the fifth plug 50 and the fourth plug 40 are discharged in order from the end on the side of the fifth plug 50 of the tube 200. The third plug 30 (or the seventh plug 70) may be discharged. However, the second plug 20 (or the sixth plug 60) is not permitted to be discharged. In this case, for example, when the volume of the third plug 30 (or the seventh plug 70) is set to be larger than those of other plugs and the length of the third plug 30 (or the seventh plug 70) in the longitudinal direction of the tube 200 is increased, the second plug 20 (or the sixth plug 60) is easily prevented from being discharged.

The fourth plug 40 and the fifth plug 50 are discharged to, for example, a reaction container for PCR. Therefore, the eluate and the wax or oil are dispensed to the reaction container for PCR. Usually, the wax or oil has no influence on PCR, and thus for example, a wax or an oil of the same kind as the wax or the like of the fifth plug 50 may be accommodated in advance in the reaction container of PCR. In that case, when this process is performed in a state in which the tip end of the tube 200 is in the wax or oil, the eluate containing the target nucleic acid can be introduced to the reaction container of PCR without the contact of the eluate with the outside air. When the nucleic acid extraction method of this embodiment includes this process, the eluate containing the target nucleic acid can be easily dispensed to, for example, a reaction container for PCR.

### 3.8. Modification Examples

### 3.8.1. Modification of Process of Moving Magnetic Particles

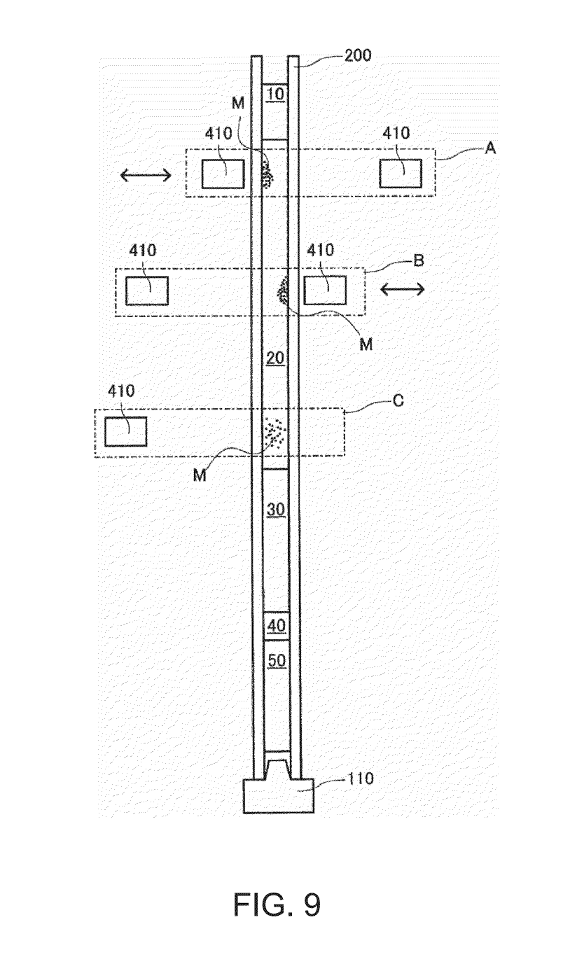

FIG. 9 is a schematic diagram for illustrating a modification of the nucleic acid extraction method of this embodiment.

In the above-described clause “3.4. Process of Moving Magnetic Particles”, the magnetic particles M pass through the plugs from the first plug 10 and are moved up to the fourth plug 40 by applying a magnetic force to the magnetic particles M from the outside. However, when the magnetic particles M are moved to the second plug 20, the magnetic particles M may be vibrated in the second plug 20, or may be repeatedly diffused and aggregated by changing the magnetic force applied from the outside. Thus, the effect of washing the magnetic particles M with the first washing liquid of the second plug 20 can be increased.

Specifically, as shown in the boxes A and B of FIG. 9, in a case in which a pair of permanent magnets 410 is used as a unit which applies a magnetic force, when the magnetic particles M are moved from the container 120, pass through the first plug 10, and reach the second plug 20 using the permanent magnets 410, the magnetic particles M can be vibrated in a direction crossing the longitudinal direction of the tube 200 in the second plug 20 when one permanent magnet 410 is moved away from the tube 200 and the other permanent magnet 410 is moved closer from the side opposed to the tube 200 (repetition of the aspects denoted by A and B of FIG. 9). Thus, the effect of washing the magnetic particles M with the first washing liquid of the second plug 20 can be increased. When the second plug 20 is partitioned or the sixth plug 60 is disposed in the tube 200, such washing of the magnetic particles M may also be applied in a plurality of second plugs 20 or in the sixth plug 60.

In addition, as shown in the box C of FIG. 9, the magnetic particles M can be diffused in the second plug 20 by simply moving the permanent magnet 410 away from the tube 200. Since the magnetic particles M have a hydrophilic surface, the magnetic particles M have difficulty entering the waxes of the first plug 10 and the third plug 30 even when, for example, the magnetic force is weakened and diffused in the second plug 20. Thus, such an aspect may be employed.

Specifically, when the magnetic particles M are moved from the container 120, pass through the first plug 10, and reach the second plug 20 using the permanent magnet 410, the permanent magnet 410 is moved away from the tube 200 to diffuse the magnetic particles M in the second plug 20. The magnetic particles M can be moved again, pass through the third plug 30, and be introduced to the fourth plug 40 using the magnetic force of the permanent magnet 410.

The aspect in which the magnetic particles M are vibrated, or repeatedly diffused and aggregated by changing the magnetic force applied from the outside may also be applied when the magnetic particles M exist in the adsorbent liquid in the container 120 or when the magnetic particles M exist in the fourth plug 40 (eluate).

### 3.8.2. Modification of Process of Eluting Nucleic Acid

In the above-described clause “3.5. Process of Eluting Nucleic Acid”, the fourth plug 40 may be heated. Examples of the method of heating the fourth plug 40 include a method of bringing a heating medium such as a heating block into contact with a position corresponding to the fourth plug 40 of the tube 200, a method using a heat source such as a heater, and an electromagnetic heating method.

When the fourth plug 40 is heated, a plug other than the fourth plug 40 may be heated. However, in a state in which the magnetic particles M adsorbing the nucleic acid exist in the washing liquid plug, the plug is preferably not heated. The temperature which is reached when the fourth plug 40 is heated is preferably 35° C. to 85° C., more preferably 40° C. to 80° C., and even more preferably 45° C. to 75° C. from the viewpoint of elution efficiency and from the viewpoint of suppression of, when the eluate contains an enzyme for PCR, deactivation of the enzyme.

In the process of eluting the nucleic acid, when the fourth plug 40 is heated, the nucleic acid adsorbed to the magnetic particles M can be more efficiently eluted to the eluate. Even when the first or second washing liquid has a composition which is the same as or similar to the composition of the eluate, the nucleic acid remaining on and adsorbed to the magnetic particles M without being eluted to the washing liquid can be eluted to the eluate. That is, even after the magnetic particles M adsorbing the nucleic acid are washed with the first or second washing liquid, the nucleic acid can be further eluted to the eluate. Accordingly, sufficient washing and elution to the eluate at a sufficient concentration can be balanced even when the composition of the washing liquid and the composition of the eluate are the same as or similar to each other.

3.8.3. Modification of Process of Discharging Fourth Plug from Tube

When the above-described “3.7. Process of Discharging Fourth Plug from Tube” is employed, the magnetic particles M from which the adsorbed nucleic acid has been eluted to the eluate in the related process may exist in the fourth plug 40, but the magnetic particles M may be moved to any one of the first plug 10, the second plug 20, and the third plug 30 or to the container 120 by applying a magnetic force. Thus, the fourth plug 40 can be discharged from the tube 200 in a state in which the eluate does not contain the magnetic particles M. When a destination of the magnetic particles M is the second plug 20 or the container 120, the magnetic particles M have difficulty entering the wax of the third plug 30 even when the magnetic force is removed, and thus the fourth plug 40 can be more easily discharged from the tube 200.

### 3.8.4. Modification Examples of Tube Portion

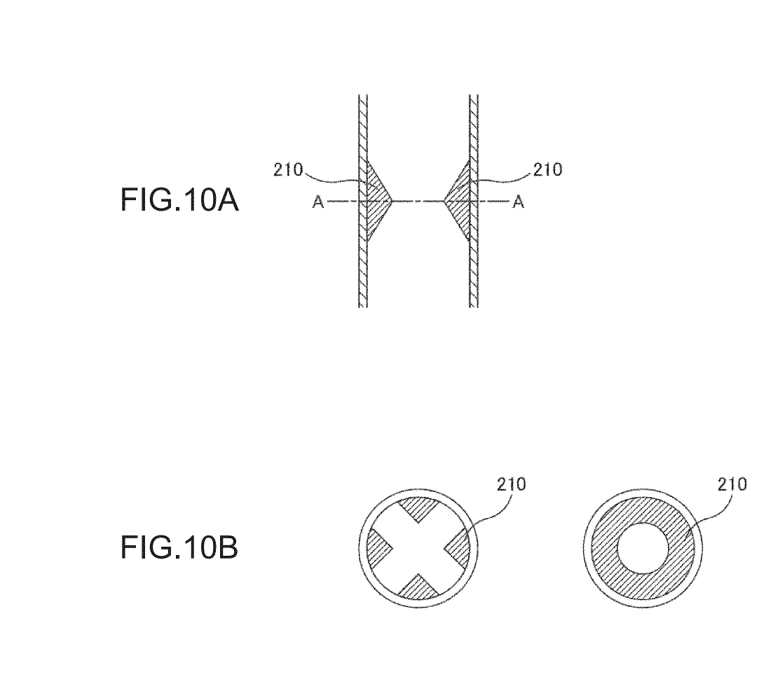

The wax is a solid at room temperature. Accordingly, when protrusions 210 are provided on the wall surface of the tube 200 as illustrated in FIG. 10A, the solid wax does not easily move in the tube 200, and thus plug deviation can be prevented. The height of a top of the protrusion 210 is not particularly limited, but is preferably approximately 1/10 to ¼ of the internal diameter of the tube. The shape of the protrusion is not particularly limited. The protrusion may have a conical shape as illustrated on the left side of FIG. 10B, or an annular shape along the internal surface of the tube as illustrated on the right side of FIG. 10B.

### 4. Nucleic Acid Extraction Apparatus

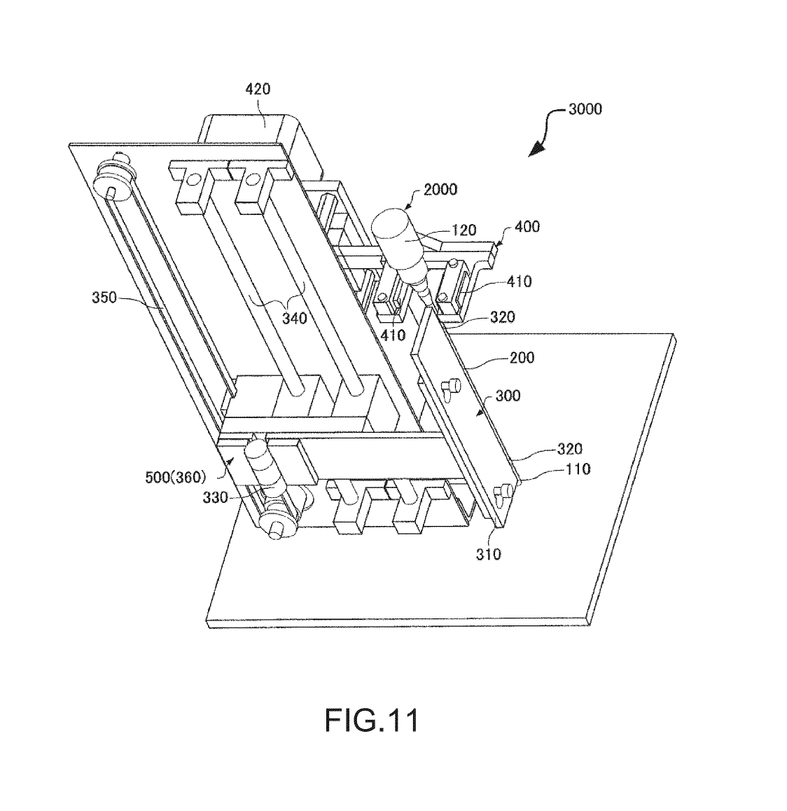

A nucleic acid extraction apparatus according to this embodiment can be appropriately applied to the nucleic acid extraction device, the nucleic acid extraction kit, and the nucleic acid extraction method which have been described above. Hereinafter, a nucleic acid extraction apparatus 3000 which extracts a nucleic acid with the nucleic acid extraction kit 2000 mounted thereon will be described as an embodiment. FIG. 11 is a perspective view schematically illustrating the nucleic acid extraction apparatus 3000 of this embodiment.

The nucleic acid extraction apparatus 3000 of this embodiment includes: a mounting portion 300 on which a tube having a longitudinal direction in which a first plug 10 formed of a wax or an oil, a second plug 20 formed of a first washing liquid which does not mix with a wax or an oil, a third plug 30 formed of a wax, a fourth plug 40 formed of an eluate which does not mix with a wax or an oil, and a fifth plug 50 formed of a wax or an oil are arranged in this order is mounted; a magnetic force application portion 400 which applies, when a tube 200 is mounted on the mounting portion 300, a magnetic force from a side surface of the tube 200; and a moving mechanism 500 which changes the relative arrangement between the mounting portion 300 and the magnetic force application portion 400 in the longitudinal direction of the tube 200.

The tube 200 mounted on the mounting portion 300 of the nucleic acid extraction apparatus 3000 is the above-described tube 200. The nucleic acid extraction apparatus 3000 has the mounting portion 300 on which the tube 200 is mounted. Although an example has been shown in which the plugs ranging from the first plug 10 to the fifth plug 50 are arranged in the tube 200, the above-described sixth plug 60 and seventh plug 70 may also be arranged.

The mounting portion 300 is a portion in which the tube 200 is mounted. Together with the tube 200, a container 120 connected to the tube 200 may also be mounted on the mounting portion 300. As the mounting portion 300, a mechanism or the like for configuration or mounting can be appropriately designed within a range in which the magnetic force application portion 400 can apply a magnetic force to the tube 200, and if necessary, to the container 120. The mounting portion 300 may be configured so that when the tube 200 is flexibly bent, the tube 200 can be mounted by being stretched into a linear shape. In addition, in the example illustrated in FIG. 11, the mounting portion 300 has a doubling plate 310 disposed along the tube 200. The doubling plate 310 is not an essential configuration. However, in some cases, vibration of the tube 200 can be suppressed when the doubling plate 310 is installed. In the example illustrated in FIG. 11, the mounting portion 300 has clip mechanisms 320, and thus the tube 200 is fixed at two places.

The mounting portion 300 is configured so that the positional relation with the magnetic force application portion 400 is relatively changed in the longitudinal direction of the tube 200. Accordingly, when a design in which the mounting portion 300 is relatively moved with respect to the magnetic force application portion 400 with no movement of the magnetic force application portion 400 is provided, a moving mechanism 360 which moves the mounting portion 300 is included as the moving mechanism 500 as illustrated in FIG. 11. In some cases, the moving mechanism 360 is not required for the mounting portion 300 when the magnetic force application portion 400 includes a moving mechanism. In the example illustrated in FIG. 11, the mounting portion 300 is configured to include a hinge 330, guide rails 340, a drive belt 350, and a motor (not illustrated).

In the example of the nucleic acid extraction apparatus 3000, one mounting portion 300 is provided, but more than one mounting portion 300 may be provided. In that case, more than one magnetic force application portion 400 can be provided, and the plural mounting portions 300 may be provided separately or in conjunction with each other.

The magnetic force application portion 400 is a configuration for applying a magnetic force to the tube 200, and if necessary, to the container 120 when the tube 200 is mounted on the mounting portion 300. The magnetic force application portion 400 is configured to include, for example, a permanent magnet, an electromagnet, or a combination thereof. The magnetic force application portion 400 is provided with at least one magnet. However, more than one magnet may be provided. It is preferable that an electromagnet not be used, but a permanent magnet be used in the magnetic force application portion 400 since generation of heat and the like are difficult to occur. As the permanent magnet, for example, a nickel-based, iron-based, cobalt-based, samarium-based, or neodymium-based permanent magnet can be used.

The magnetic force application portion 400 functions to apply a magnetic force to magnetic particles M which exist in the container 120 and in the tube 200. The magnetic particles M can be moved in the container 120 and in the tube 200 by changing the relative positional relation between the mounting portion 300 and the magnetic force application portion 400.

In the example illustrated in FIG. 11, the magnetic force application portion 400 has a pair of permanent magnets 410 provided opposed to each other with the container 120 and the tube 200 interposed therebetween. The pair of permanent magnets 410 is separated from each other at an interval larger than an external diameter of the tube 200. The direction of the polarity of the permanent magnet 410 is not particularly limited. The magnetic force application portion 400 is configured so that the positional relation with the mounting portion 300 is relatively changed in the longitudinal direction of the tube 200. Accordingly, when a design in which the magnetic force application portion 400 is relatively moved with respect to the mounting portion 300 with no movement of the mounting portion 300 is provided, a moving mechanism which moves the magnetic force application portion 400 is included as the moving mechanism 500.

In addition, in the example illustrated in FIG. 11, the magnetic force application portion 400 is disposed so that when one of the pair of permanent magnets 410 is moved closer to the tube 200, the other one is separated from the tube 200. Vibration can be applied using a motor 420 so that the pair of permanent magnets 410 is moved closer to or separated from the tube 200. The magnetic particles M can be moved to reciprocate in a direction crossing the longitudinal direction of the tube 200 in the tube 200 by driving the motor 420.

If necessary, the motor 420 can also be driven when a magnetic force is applied to any of the container 120 and the tube 200. Efficiency of washing the magnetic particles M in the tube 200 or elution efficiency can be increased when the motor 420 is driven at the time when the permanent magnet 410 is positioned at the position of the second plug 20 or the position of the fourth plug 40 of the tube 200.

According to the nucleic acid extraction apparatus 3000 of this embodiment, preprocessing for PCR can be automated, and the time and effort required for the preprocessing can be significantly reduced. In addition, according to the nucleic acid extraction apparatus 3000 of this embodiment, since the magnetic force application portion 400 can be vibrated, washing (purification) of magnetic particles M adsorbing a nucleic acid can be more efficiently performed, and thus the accuracy of PCR can be further increased.

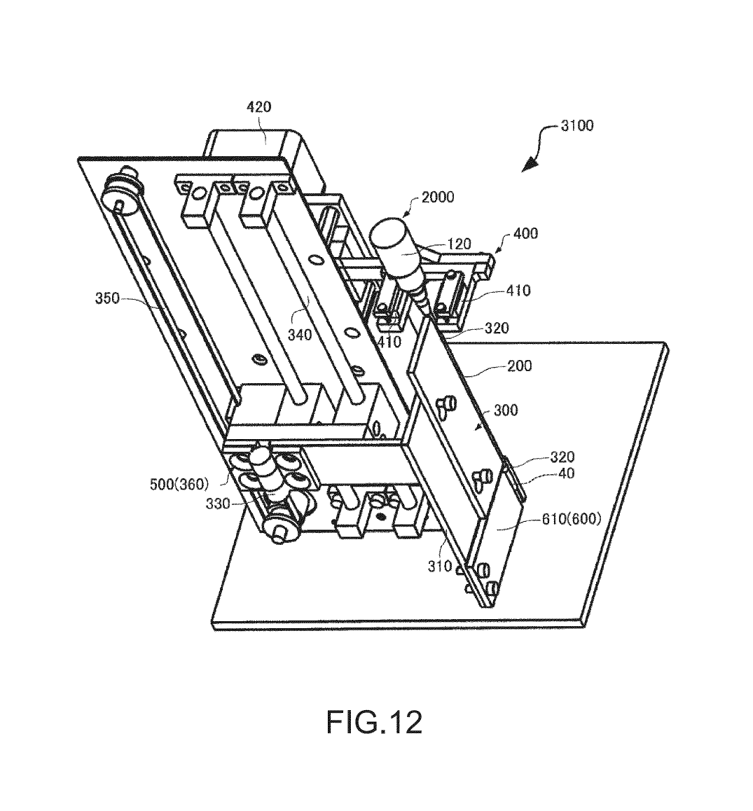

FIG. 12 is a perspective view schematically illustrating a nucleic acid extraction apparatus 3100 according to a modification example of the nucleic acid extraction apparatus. The nucleic acid extraction apparatus 3100 is the same as the above-described nucleic acid extraction apparatus 3000, except that a heating portion 600 is provided. Members having common actions and functions will be denoted by the same reference numerals, and description thereof will be omitted.

The heating portion 600 is configured to heat a part of the tube 200 when the tube 200 is mounted on the mounting portion 300. Examples of the heating portion 600 include a heat source, a heating block, a heater, and a coil for electromagnetic heating. The heating portion 600 is shaped to allow insertion of the tube 200 therein or to be brought into contact with the side surface of the tube 200, and is arbitrarily shaped as long as the liquid in the tube 200 can be heated.

The part heated by the heating portion 600 in the tube 200 includes a part where the third plug 30 in which a wax is disposed exists in the longitudinal direction of the tube 200. A part where the fourth plug 40 exists may be included therein. The heating portion 600 may heat another part of the tube 200 including the part where the fourth plug 40 exists.

The nucleic acid extraction apparatus 3100 illustrated in FIG. 12 is provided with, as the heating portion 600, a heater 610 which is provided in parallel to the doubling plate 310 to heat a position including the fourth plug 40 of the tube 200. The heater 610 is shaped to be brought into contact with approximately half of the outer periphery of the tube 200.

The nucleic acid extraction apparatus 3100 can elute a sufficient amount of a nucleic acid to the eluate of the fourth plug 40 even when the amount of the nucleic acid adsorbed to magnetic particles M is reduced by washing with at least one of the first washing liquid of the second plug 20 and the second washing liquid of the sixth plug 60. Accordingly, the washing effect can be increased, and a sufficient concentration of a nucleic acid for PCR can be eluted to the eluate.

### EXAMPLES

Hereinafter, examples will be described to describe the invention in further detail. However, the invention is not limited to the following examples.

### Experimental Example 1

A hollow polypropylene tube having an internal diameter of 1 mm was filled with ethanol (25 μL), a barrier layer (25 μL), and pure water (1 μL) in order. As the barrier layer, a silicone oil (manufactured by Shin-Etsu Chemical Co., Ltd.) having a kinematic viscosity of 2 cSt (25° C.), a mineral oil (manufactured by Sigma Corporation), or a solid paraffin (manufactured by Wako Pure Chemical Industries, Ltd.) having a melting point of 60° C. to 62° C. was used. Both ends of the filled tube were sealed using cocks and the tube was left at 30° C. Thereafter, it was preserved for one, two, and six weeks to evaluate whether the ethanol mixed with the pure water through the barrier layer.

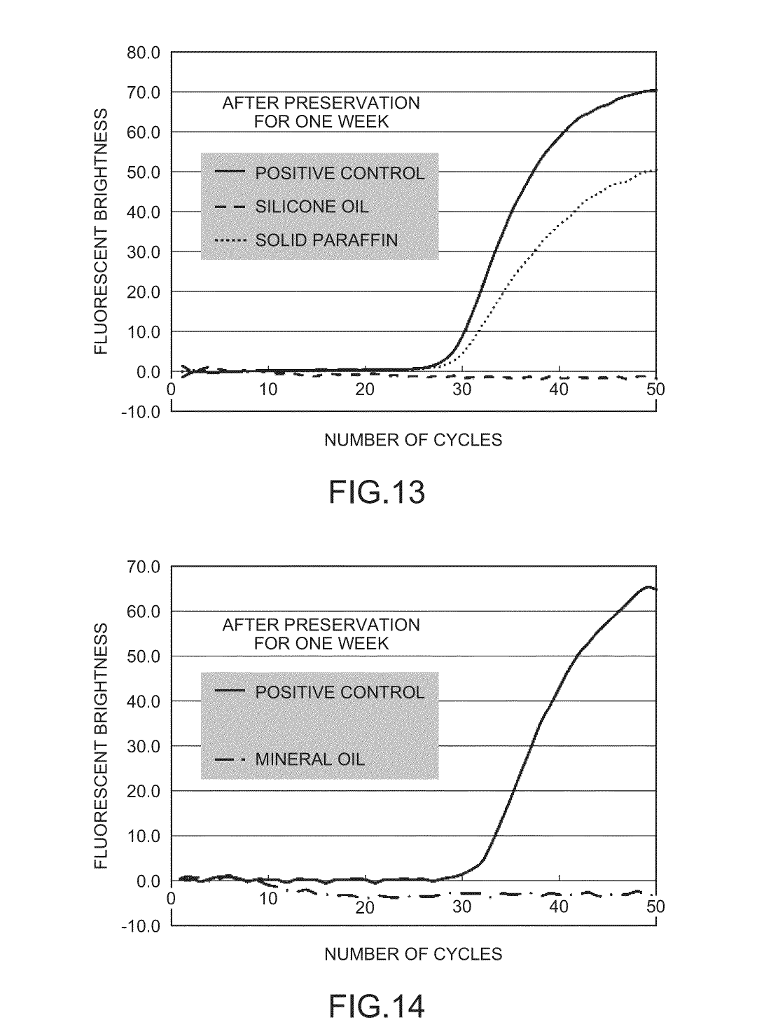

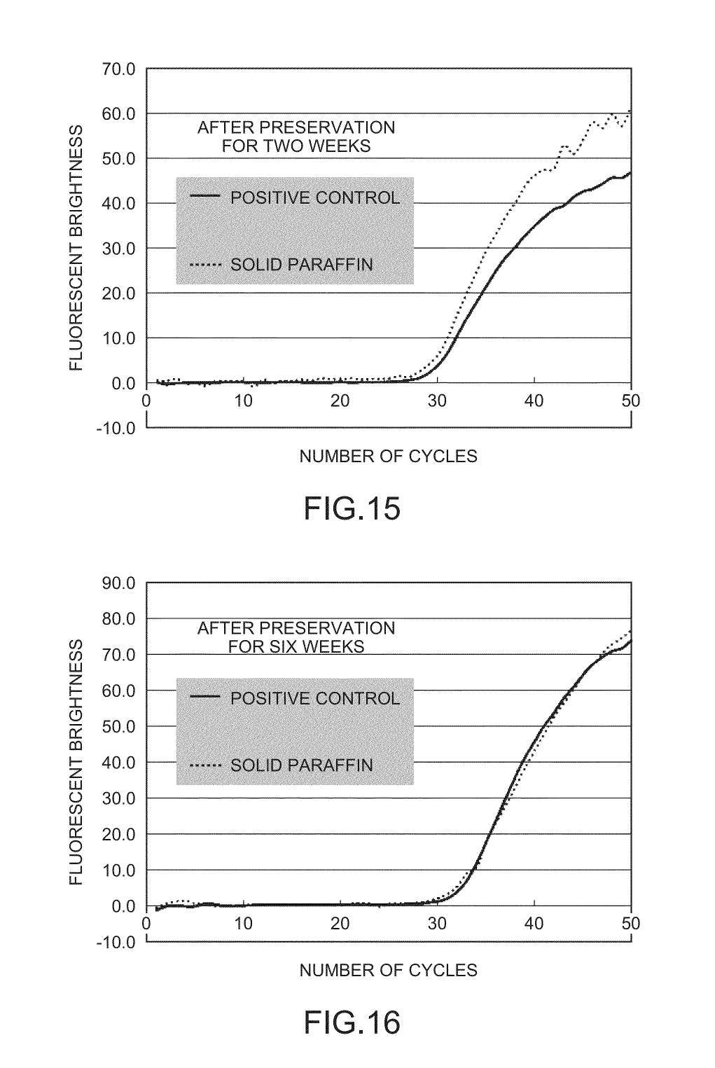

As the evaluation method, a phenomenon was used in which the ethanol inhibits RT-PCR. That is, only the pure water part in the tube was extracted after preservation for a predetermined time, and was added to and mixed well with a freeze-dried RT-PCR reagent prepared according to the rule. Thereafter, influenza A virus RNA extracted and purified in advance was added thereto to perform RT-PCR. RT-PCR was carried out in the same manner as in the method described in Clinica Chimica Acta 413 (2012) 1742-1745. The results after preservation of the silicone oil and the solid paraffin for one week are illustrated in FIG. 13, the results after preservation of the mineral oil for one week are illustrated in FIG. 14, the results after preservation of the solid paraffin for two weeks are illustrated in FIG. 15, and the results after preservation of the solid paraffin for six weeks are illustrated in FIG. 16. The positive control represents results of using pure water prepared before use.

As a result, in the cases of the silicone oil and the mineral oil, the ethanol was transmitted therethrough and dissolved in the pure water when the tube was preserved for one week at 30° C. However, when the solid paraffin was used as a barrier layer, it was possible to prevent the transmission of the ethanol even when the tube was preserved for six weeks at 30° C.

When using the solid paraffin as a barrier layer, stable preservation is possible in a state in which, even when one hollow tube is filled with preprocessing reagents in advance and preserved for a long time, the respective reagent components have desired functions without being dispersed.

### Example 2

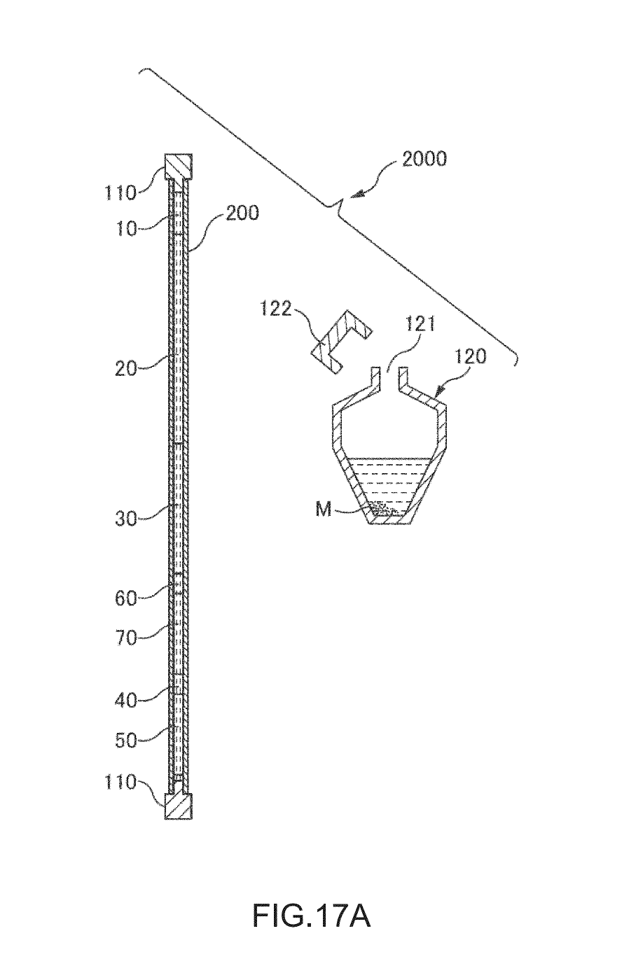

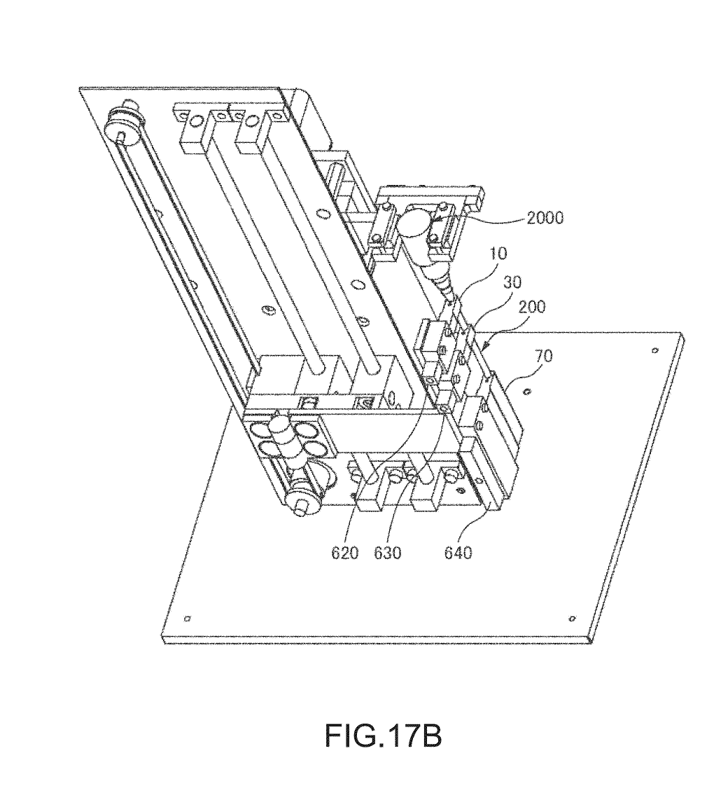

In Example 2, a nucleic acid extraction apparatus (FIG. 17B) was used, and preprocessing was performed with a nucleic acid extraction device (FIG. 17A) using a solid paraffin.

A first plug 10, a third plug 30, a fifth plug 50, and a seventh plug 70 are each formed of a solid paraffin (manufactured by Wako Pure Chemical Industries, Ltd.) having a melting point of 60° C. to 62° C. A second plug 20 is a first washing liquid (70% ethanol). A sixth plug 60 is a second washing liquid (1 mM citric acid buffer; pH 4.0). A fourth plug 40 is an eluate (pure water).

First, in order to liquify the solid paraffins, a tube 200 of the nucleic acid extraction device was set on heaters 620, 630, and 640 capable of heating the first plug 10, the third plug 30, the fifth plug 50, and the seventh plug 70 illustrated in FIG. 17B, and the plugs were heated at 82° C.

Next, 700 μL of an adsorbent liquid and 2 μL of a magnetic bead dispersion liquid were accommodated in a polyethylene container having a capacity of 3 mL. The adsorbent liquid had a composition of 3 M of guanidinium thiocyanate, 1 mass % of polyoxyethylene sorbitan monolaurate, 25 mM of Tris-HCl (pH 7.2), and a 50%-ethanol aqueous solution. A solution having a concentration of 400 mg/mL was used as the magnetic bead dispersion liquid.

150 μL of a solution containing an influenza A positive sample (nasal cavity wiping liquid) was put from an opening 121 of the container 120 using a pipette. The container 120 was covered with a lid and shaken for stirring for 30 seconds by hand. Thereafter, the lid 122 of the container 120 was removed and the container was connected to the tube 200. The tube 200 had cocks 110 mounted at both ends thereof, and the cock on the side of the first plug was removed to connect the container 120 to the tube 200.

The magnetic beads M in the container were introduced into the tube 200 by moving a permanent magnet by hand. The magnetic beads were vibrated and moved up to the sixth plug. Times for which the magnetic beads M existed in the respective plugs in the tube 200 were roughly as follows: first, third and fifth plugs: 3 seconds; second plug: 20 seconds; fourth plug: 30 seconds; and sixth plug: 20 seconds. An operation of vibrating the magnetic beads was not performed in the seventh plug. The volumes of the second plug, the sixth plug, and the fourth plug were 25 μL, 25 μL, and 1 μL, respectively.

Next, the magnetic beads were moved and retreated up to the second plug using the permanent magnet, and then the cock on the side of the seventh plug of the tube was removed and the container was deformed by hand to discharge the fourth plug and the fifth plug to the container.

Next, real-time RT-PCR was performed. The RT-PCR was carried out according to CDC protocol of real-time RTPCR for swine influenza A (H1N1). That is, 9 μL of a solution containing: 0.8 μM of a forward primer; 0.8 μM of a reverse primer; 0.2 μM of a probe; 1× SuperScript III RT/Platimun Taq Mix; and 1× PCR Master Mix was prepared, and 1 μL of the eluate discharged a while ago was added thereto. After stirring, the RT-PCR was performed under the following protocols.
Reverse Transcription: 50° C., 30 minutes
Taq Activation: 95° C., 2 minutes
PCR: 95° C., 15 seconds-55° C., 30 seconds; 50 cycles

Primer:
Primer F:
GAC CAA TCC TGT CAC CTC TGA C
Primer R:
AGG GCA TTT TGG ACA AAG CGT CTA
Probe:
TaqMan probe:
FAM-TGC AGT CCT CGC TCA CTG GGC ACG-TAMRA

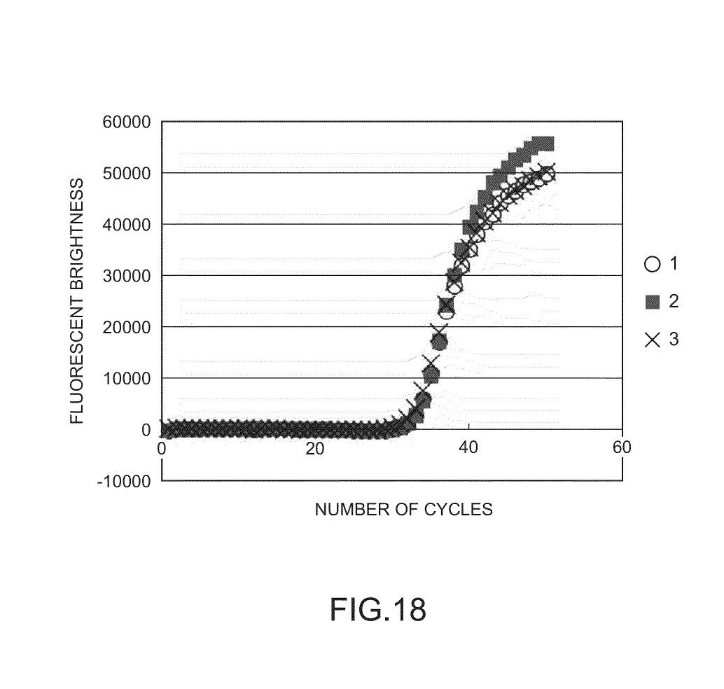

FIG. 18 illustrates results of the experiment performed with n=3. From FIG. 18, it is found that using this apparatus, the RNA of the influenza A virus is extracted with no problems and is detected through the RT-PCR.

The entire disclosure of Japanese Patent Application No. 2013-223011 filed Oct. 28, 2013 is expressly incorporated by reference herein.

What is claimed is:

1. A nucleic acid extraction device comprising:
a tube extending in a longitudinal direction from an upstream end to a downstream end;
a first plug provided in the tube proximate the upstream end, the first plug being formed of wax or oil;
a second plug provided in the tube downstream of the first plug, the second plug being formed of a first washing liquid;
a third plug provided in the tube downstream of the second plug, the third plug being formed of wax;
a fourth plug provided in the tube downstream of the third plug, the fourth plug being formed of an eluate; and
a fifth plug provided in the tube downstream of the fifth plug, the fifth plug being formed of wax or oil.

2. The nucleic acid extraction device according to claim 1, further comprising:
a sixth plug provided in the tube downstream of the third plug and upstream of the fourth plug, the sixth plug being formed of a second washing liquid; and
a seventh plug provided in the tube downstream of the sixth plug and upstream of the fourth plug, the seventh plug being formed of wax or oil.

3. The nucleic acid extraction device according to claim 2,
wherein the seventh plug is wax.

4. The nucleic acid extraction device according to claim 1,
wherein the wax of at least one of the first, third and fifth plugs is a paraffin that is solid at room temperature.

5. The nucleic acid extraction device according to claim 1,
wherein the wax of at least one of the first, third and fifth plugs has a melting point of 41° C. or higher.

6. The nucleic acid extraction device according to claim 1,
wherein the first plug and the fifth plug are wax.

7. The nucleic acid extraction device according to claim 1,
wherein the tube has an opening at the downstream end.

8. The nucleic acid extraction device according to claim 7, further comprising:
a detachable cock which seals the opening.

9. The nucleic acid extraction device according to claim 1, further comprising:
a detachable container at the upstream end of the tube,
wherein an inside of the container fluidly communicates with an inside of the tube.

10. The nucleic acid extraction device according to claim 9,
wherein the container is flexible, and
wherein the inside of the tube is pressurized by deforming the container while the container is connected to the tube.

11. The nucleic acid extraction device according to claim 1, further comprising:
a liquid reservoir at the upstream end of the tube.

12. The nucleic acid extraction device according to claim 11, further comprising:
a detachable cock which seals an opening of the liquid reservoir.

13. The nucleic acid extraction device according to claim 12,
wherein the liquid reservoir is flexible, and
wherein an inside of the tube is pressurized by deforming the liquid reservoir while the opening is sealed with the cock.

14. The nucleic acid extraction device according to claim 1,
wherein the eluate contains at least one of the group consisting of a reverse transcription enzyme, dNTP, and a primer for reverse transcription.

15. The nucleic acid extraction device according to claim 1,
wherein the eluate has a volume of 0.5 μL to 3.0 μL.

16. The nucleic acid extraction device according to claim 1,
wherein the tube has an internal diameter of 0.5 mm to 3.0 mm.

17. A nucleic acid extraction device comprising:
a tube extending in a longitudinal direction;
a first plug provided in the tube, the first plug being formed of wax or oil; and
a second plug provided in the tube, the second plug being formed of an aqueous solution.

18. The nucleic acid extraction device according to claim 17,
wherein the tube has an internal diameter of 0.5 mm to 3.0 mm.

19. A nucleic acid extraction device comprising:
a tube extending in a longitudinal direction;
a plurality of wax or oil plugs provided in the tube; and
a plurality of aqueous plugs provided in the tube alternately with the plurality of wax or oil plugs.

20. The nucleic acid extraction device according to claim 19,
wherein the tube has an internal diameter of 0.5 mm to 3.0 mm.


SEQUENCE LISTING (3 sequences)

SEQ ID NO: 1 - DNA, 22 bp, Influenza A virus
gaccaatcctgtcacctctgac

SEQ ID NO: 2 - DNA, 24 bp, Influenza A virus
agggcattttggacaaagcgtcta

SEQ ID NO: 3 - DNA, 24 bp, Influenza A virus
tgcagtcctcgctcactgggcacg